(19) United States
(12) Patent Application Publication
(10) Pub. No.: US 2011/0034678 A1
(43) Pub. Date: Feb. 10, 2011

(54) METHODS OF RENATURATION OF RECOMBINANT PROTEINS

(75) Inventors: Wayne Froland, Alameda, CA (US); Katherine Delaria, Walnut Creek, CA (US); David Boisvert, El Cerrito, CA (US); Robert Kuhn, Corte Madera, CA (US)

Correspondence Address: DLA PIPER LLP (US), 4365 EXECUTIVE DRIVE, SUITE 1100, SAN DIEGO, CA, 92121-2133, US

(73) Assignee: AEROVANCE, INC., Berkeley, CA (US)

(21) Appl. No.: 12/723,517

(22) Filed: Mar. 12, 2010

Related U.S. Application Data
(60) Provisional application No. 61/160,249, filed on Mar. 13, 2009.

(51) Int. Cl. C07K 1/107; C07K 1/16

(52) U.S. Cl. 530/408; 530/413

(57) ABSTRACT

The present invention provides large-scale methods for renaturation of proteins comprising adding a solution of denatured, chemically modified or reduced proteins to a refolding buffer containing sulfate derived from H₂SO₄ and/or MgSO₄ in the presence of guanidine. The present invention further provides methods of isolating a refolded protein at a concentration of about 0.4 to 3.0 gm/L by using a hydrophobic interaction chromatography (HIC) column.

### CROSS REFERENCE TO RELATED APPLICATION(S)

This application claims the benefit of priority under 35 U.S.C. §119(e) of U.S. Ser. No. 61/160,249, filed Mar. 13, 2009, the entire content of which is incorporated herein by reference.

### BACKGROUND OF THE INVENTION

1. Field of the Invention

The invention relates to general methods for the renaturation of recombinant, eukaryotic proteins containing disulfide bonds after expression in prokaryotes, and more specifically to the use of hydrophobic interaction chromatography (HIC) for renatured protein capture in large-scale methods thereof.

2. Background Information

Production of recombinant proteins in heterologous expression systems like e.g., Escherichia coli, often yields inactive, insoluble aggregates (so-called “refractile bodies” or “inclusion bodies”). Preparations of these inclusion bodies are contaminated by host cell components like host cell proteins, nucleic acids, endotoxins and low molecular weight impurities. It is assumed that the formation of inclusion bodies is a result of very high local concentrations of the heterologous protein in the cell during induction and protein biosynthesis. However, the primary amino acid sequence of the heterologous protein in question is also of great importance, particularly the presence of cysteine-residues that form covalent disulfide bonds during oxidative refolding. Before these target proteins can be used, e.g., for therapeutic purposes, the inclusion bodies have to be purified, solubilized and, subsequently, the recombinant protein native three-dimensional structure has to be renatured to convert (refold) the protein into its biologically active conformation.

A commonly applied sequence of process steps involves, first, the solubilization of the inclusion bodies by the addition of high concentrations of chaotropic, denaturing agents (e.g., guanidinium hydrochloride or urea), or by the addition of strongly acidic agents (e.g., glycine/phosphoric acid mixtures). Concurrently, intramolecular and intermolecular disulfide bonds present in the inclusion bodies may be either reduced chemically or cleaved by the so-called sulfitolysis procedure involving sulfite and an oxidizing agent. Secondly, the solubilized protein mixture may be further purified by chromatographic and/or filtration methods, both of which are well known procedures for those skilled in the art.

Typically, the linearized monomeric protein solution in the presence of high concentrations of a chaotropic agent is highly diluted in order to allow for the formation of the biologically active form. This can be performed either rapidly (by simple dilution into a large volume of refolding buffer) or slowly by very gradual dilution into a large volume of refolding buffer or by diafiltration or dialysis against the refolding buffer. Other techniques described in the literature involve the adsorption of the target protein onto a chromatographic resin and, subsequently, lowering the concentration of chaotropic agent allowing refolding to take place; or size exclusion chromatography in order to resolve the protein chains from the low molecular weight chaotropic agent. In every case, the concentration of the chaotropic salt has to be decreased below a certain limit that is dependent on the target protein, e.g., usually below 0.5 M guanidinium hydrochloride.

The major side reaction during refolding is the formation of soluble and insoluble aggregates of misfolded intermediates that is dependent on the local concentration of folding intermediates. In the literature, a broad range of folding aids are described, effectively suppressing the formation of soluble and insoluble protein aggregates, like for example, chaperone proteins, other types of proteins (e.g., bovine serum albumin), and several types of non-protein materials, including sugars and cyclic sugars, amino acids, short chain alcohols (e.g., glycerol, pentanol, hexanol), enzyme substrates, synthetic polymers, detergents, and chaotropic salts.

U.S. Publication No. 20050014933, incorporated herein by reference, describes a different approach for renaturation of proteins involving use of a refolding buffer containing tris(hydroxymethyl)-aminomethane (Tris) in combination with sulfate (SO₄²⁻) from sulfuric acid at high concentrations to affect solubilization of folding intermediates to increase the refolding yield while increasing purity. However, preparation of Tris-SO₄ by adding sulfuric acid to Tris-base poses hazards at the larger manufacturing scale.

Hence, there is still a need to develop strategies for protein refolding using conventional techniques. From the state of the art, no generally useable, chemically simple and inexpensive aggregation suppressor is known that can be applied in a commercially attractive, high yield, refolding process of proteins at high protein concentrations of up to 2.0 gm/L.

### SUMMARY OF THE INVENTION

Starting from insoluble protein aggregates (so-called inclusion bodies) as obtained by overexpression of recombinant protein in Escherichia coli, the present invention provides methods of large-scale renaturation of proteins like, e.g., Interleukin-4 (IL-4) and muteins thereof, at high protein concentrations. Exemplary IL-4 muteins include, but are not limited to, proteins having an amino acid sequence as set forth in SEQ ID NO: 1, 2, or 3.

As such, in one embodiment, the invention provides a method for large-scale renaturation of proteins. In one embodiment, the method includes adding to a refolding buffer a protein solution of about 12 to 20 gm/L of denatured, chemically modified or reduced protein in the presence of guanidine, wherein the refolding buffer comprises a Tris-base/Tris-HCl system. In another embodiment, the method includes adding to a refolding buffer a solution of about 12 to 20 gm/L of denatured, chemically modified or reduced protein in the presence of guanidine, wherein the refolding buffer includes MgSO₄ in a Tris-base/Tris-HCl system. In another embodiment, the method includes adding to a refolding buffer a solution of about 10 to 35 gm/L of denatured, chemically modified or reduced protein in the presence of guanidine to a diluted protein concentration of 0.25 to 3.0 gm/L in refolding buffer, wherein the refolding buffer includes sulfate (SO₄²⁻) in a Tris-base/Tris-HCl buffer system. In one embodiment, the refolding buffer includes 0 M to 1.0 M H₂SO₄ or 0.1 M to 0.6 M MgSO₄, 0.1 M to 1.0 M Tris Base, 0.1 M to 1.0 M Tris HCl, 2 mM to 7 mM EDTA, 0.5 mM to 2 mM cysteine, and protein concentration of 0.25 to 3.0 gm/L. In another embodiment, the refolding buffer includes 0.2 M to 0.6 M MgSO₄, 0.1 M to 0.6 M Tris Base, 0.7 M to 1.4 M Tris HCl, 2 mM to 7 mM EDTA, and 0.5 mM to 2 mM cysteine. In another embodiment, the refolding buffer includes 0 M to 1.0 M H₂SO₄ or 0.1 M to 0.6 M MgSO₄, 0.1 M to 1.0 M Tris Base, 0.1 M to 1.0 M Tris HCl, 2 mM to 7 mM EDTA, 0.5 mM to 2 mM cysteine, and protein concentration of 0.4 to 2.0 gm/L. In another embodiment, the refolding buffer includes 0.4M MgSO₄, and further comprises 0.125M Tris Base, 0.875M Tris HCl, 5 mM EDTA, and 1 mM cysteine. In yet another embodiment, the refolding buffer includes 0.4M H₂SO₄, and further comprises 0.125M Tris HCl, 0.875M Tris Base, 5 mM EDTA, 1 mM cysteine and protein concentration of 1.5 gm/L. In another embodiment, the refolding buffer further includes 0.25 mM beta-mercaptoethanol and/or 5%-20% sucrose.

The denatured protein can be added in any of a variety of methods. In one embodiment, the addition of the denatured protein occurs via pulsed dilution, such as 1 to 3 dilutions with 0 to 4 hours between dilutions. In another embodiment, the denatured protein is added in 3 dilutions with 4 hours between dilutions.

The denatured protein may further be diafiltered with 1 to 10 diavolumes of a diafiltration buffer prior to addition to the refolding buffer, wherein the diafiltration buffer can include 3 M to 7 M guanidine, 25 mM to 75 mM Tris, and 1 mM to 7 mM EDTA, at pH 7.0 to pH 9.0. In one embodiment, the diafiltration buffer includes 50 mM Tris, and 2 mM EDTA at a pH of 9.0. In another embodiment, the diafiltration buffer includes 4 M guanidine, 50 mM Tris, and 2 mM EDTA at a pH of 9.0. In another embodiment, the diafiltration buffer includes 4 M guanidine, 50 mM Tris, and 2 mM EDTA at a pH of 7.5. In another embodiment, the diafiltration buffer includes 4 M guanidine, 50 mM Tris, and 5 mM EDTA at a pH of 9.0. In another embodiment, the diafiltration buffer includes 4 M guanidine, 50 mM Tris, and 5 mM EDTA at a pH of 7.5. In another embodiment, the protein is diafiltered with 1 to 5 diavolumes of diafiltration buffer. In yet another embodiment, the protein is diafiltered with 5 diavolumes of diafiltration buffer. In yet another embodiment, the protein is diafiltered with 3 diavolumes of diafiltration buffer.

Once the refolding process has completed, the protein may then be recovered from the refolding buffer via hydrophobic interaction chromatography (HIC). In one embodiment, the pH is lowered to about 2.1 to 5.8 prior to HIC. In another embodiment, the pH is lowered to about 3.0 prior to HIC. In another embodiment, ammonium sulfate ((NH₄)₂SO₄) or sodium sulfate (Na₂SO₄) is added to a final concentration of 0.3 to 1.2 M to the refold hold vessel. In another embodiment, the pH of the refolding buffer is lowered to about 1.5 to 7.0 after completion of the refolding process and prior to use of HIC. In another embodiment, the pH of the refolding buffer post-refold is lowered to about 3.0 prior to use of HIC. In another embodiment, sodium sulfate (Na₂SO₄) is added to a final concentration of 0.4 to 0.8 M to the refold hold vessel and then the pH of the refolding buffer is lowered to about 2.1 to 5.8 after completion of the refolding process and prior to use of HIC. In another embodiment, sodium sulfate (Na₂SO₄) is added to a final concentration of 0.6 M to the refold hold vessel and then the pH of the refolding buffer is lowered to about 3.0 after completion of the refolding process and prior to use of HIC. In another embodiment, ammonium sulfate ((NH₄)₂SO₄) is added to a final concentration of 0.6 M to 1.2 M to the refold hold vessel and then the pH of the refolding buffer is lowered to about 2.1 to 5.8 after completion of the refolding process and prior to use of HIC. In another embodiment, ammonium sulfate ((NH₄)₂SO₄) is added to a final concentration of 0.6 M to the refold hold vessel and then the pH of the refolding buffer is lowered to about 3.0 after completion of the refolding process and prior to use of HIC.

In another aspect, the invention provides a method for large-scale renaturation of proteins. In one embodiment, the method includes diafiltering a denatured, chemically modified, or reduced protein with 1 to 10 diavolumes of a diafiltration buffer, wherein the diafiltration buffer comprises guanidine, concentrating the diafiltered protein to about 12 to 20 gm/L, and adding the concentrated protein via pulsed dilution to a refolding buffer, wherein the refolding buffer comprises a Tris-base/Tris-HCl system. In another embodiment, the method includes diafiltering a denatured, chemically modified, or reduced protein with 1 to 10 diavolumes of a diafiltration buffer, wherein the diafiltration buffer comprises guanidine, concentrating the diafiltered protein to about 12 to 20 gm/L, and adding the concentrated protein via pulsed dilution to a refolding buffer, wherein the refolding buffer comprises MgSO₄ in a Tris-base/Tris-HCl system. In another embodiment, the method includes concentrating the denatured, chemically modified, or reduced protein to about 10 to 35 gm/L and then diafiltering the concentrated denatured protein with 1 to 10 diavolumes of a diafiltration buffer, wherein the diafiltration buffer comprises guanidine, and adding the concentrated diafiltered protein via pulsed dilution to a refolding buffer, wherein the refolding buffer includes H₂SO₄/Tris-base/Tris-HCl system. In another embodiment, the refolding buffer comprises MgSO₄. In yet another embodiment, the refolding buffer comprises Tris hemisulfate.

In another aspect, the invention provides a method of isolating a refolded protein at a concentration of about 0.4 to 3 gm/L. The method includes loading a pH-adjusted protein solution to a hydrophobic interaction chromatography (HIC) column, and eluting the protein with an elution buffer.

In another aspect, the invention provides a method of isolating a refolded protein at a concentration of about 0.1 to 0.8 gm/L from an initial refolding concentration of denatured protein of 0.4 to 2.0 gm/L. The method includes loading a pH-adjusted refolding protein solution to a hydrophobic interaction chromatography (HIC) column, and eluting the protein with an elution buffer. In one embodiment, the protein solution is adjusted to a pH of about 1.5 to 7.0 prior to HIC. In another embodiment, the protein solution is adjusted to a pH of about 2.1 to 5.8 prior to HIC. In another embodiment, the protein solution is adjusted to a pH of 3.0 prior to HIC. In another embodiment, the HIC elution buffer includes 0 to 0.28 M ammonium sulfate, 8 mM to 12 mM potassium phosphate, pH 3.0. In another embodiment, the HIC elution buffer includes 0 to 0.35 M ammonium sulfate, 8 mM to 12 mM potassium phosphate, pH 3.0. In another embodiment, the elution buffer comprises 0.25 M ammonium sulfate, 10 mM potassium phosphate, pH 3.0. In another embodiment, the elution buffer includes 0.2 to 0.28 M ammonium sulfate, 8 mM to 12 mM potassium phosphate, pH 3.0. In another embodiment, the elution buffer comprises 0.25 M ammonium sulfate, 10 mM potassium phosphate, pH 3.0. In another embodiment, the elution buffer includes 0.15 to 0.3 M sodium sulfate, 8 mM to 12 mM potassium phosphate, pH 3. In another embodiment, the elution buffer comprises 0.25 M sodium sulfate, 10 mM potassium phosphate, pH 3.0. In another embodiment, the elution buffer includes sodium sulfate. In another embodiment, the elution buffer includes 0 to 0.3 M sodium sulfate, 8 mM to 12 mM potassium phosphate, pH 3. In another embodiment, the elution buffer comprises 0.25 M sodium sulfate, 10 mM potassium phosphate, pH 3.0. In another embodiment, the elution buffer comprises 10 mM potassium phosphate, pH 3.0. In another embodiment, the protein solution is adjusted to a pH of about 2.1 to 5.8 prior to HIC. In another embodiment, the protein solution is adjusted to a pH of 3.0 prior to HIC.

### BRIEF DESCRIPTION OF THE DRAWINGS

FIG. 1 is a graphical diagram showing the time course of refold reaction.

FIG. 2 is a graphical diagram showing the chromatography profile of IL-4RA elution from Butyl 650M. The elution (6CV) was collected in 2 fractions, with the main peak in the first 3 CVs.

FIG. 3 is a pictorial diagram showing the results from SDS-PAGE/silver stain and Western blot analysis of HIC protein load and elute.

### DETAILED DESCRIPTION OF THE INVENTION

The present invention is based on manufacturing and analytical methods for the renaturation and recovery of recombinant, disulfide bridged proteins after heterologous expression in prokaryotes.

Before the present methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

Unless otherwise required by context, singular terms shall include pluralities and plural terms shall include the singular.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Any section headings used herein are for organizational purposes only and are not to be construed as limiting the subject matter described. All references cited in this application are expressly incorporated by reference herein.

The expression of recombinant interleukin-4 derivatives employing Escherichia coli as host organism has been already described in detail (Apeler H, Wehlmann H, (1999) Plasmids, their construction and their use in the manufacture of Interleukin-4 and Interleukin-4 muteins, European patent application EP 00100129.6, Jan. 7, 1999).

Antagonists of IL-4 have been reported in the literature. Mutants of IL-4 that function as antagonists include the IL-4 antagonist mutein IL-4/Y124D (Kruse, N., et al., Conversion of human interleukin-4 into a high affinity antagonist by a single amino acid replacement, Embo J. 11:3237-44, 1992) and a double mutein IL-4[R121D/Y124D] (Tony, H., et al., Design of Human Interleukin-4 Antagonists in Inhibiting Interleukin-4-dependent and Interleukin-13-dependent responses in T-cells and B-cells with high efficiency, Eur. J. Biochem. 225:659-664 (1994)). The single mutein is a substitution of tyrosine by aspartic acid at position 124 in the D-helix. The double mutein is a substitution of arginine by aspartic acid at position 121, and of tyrosine by aspartic acid at position 124 in the D-helix. Variations in this section of the D helix do not prohibit binding of IL-4 to the primary subunit of the IL-4 receptor complex (IL-4Rα protein) but do positively correlate with changes in both interactions between IL-4/IL-4Rα and the common γ-chain receptor subunit and IL-4/IL-4Rα and IL-13Rα1 protein.

Mutant variants of IL-4 demonstrating agonism or antagonism of wild-type IL-4 are useful for treating conditions associated with one of the pleiotropic effects of IL-4. For instance, antagonists of IL-4 would be useful in treating conditions exacerbated by IL-4 production and signaling such as asthma, eczema, allergy, or other inflammatory response-related conditions. Agonists of IL-4 may be useful for treating conditions wherein the presence of IL-4 is associated with the amelioration or attenuation of a disease, for example, an autoimmune disease such as rheumatoid arthritis, multiple sclerosis, insulin-dependent diabetes mellitus, etc. These autoimmune diseases are characterized by a polarization in production of the T helper cell populations, types 1 and 2 (Th1, Th2). Naive CD4+ T cells differentiate into Th1 or Th2 subsets, depending on the cytokine or mixture of cytokines present during stimulation.

As used herein, “wild type IL-4” or “wtIL-4” and equivalents thereof are used interchangeably and mean human Interleukin-4, native or recombinant, having the 129 normally occurring amino acid sequence of native human IL-4, as disclosed in U.S. Pat. No. 5,017,691, incorporated herein by reference. Further, the modified human IL-4 receptor antagonists described herein may have various mutations, insertions and/or deletions and/or couplings to a non-protein polymer, and are numbered in accordance with the wtIL-4, which means that the particular amino acid chosen is that same amino acid that normally occurs in the wtIL-4. Accordingly, one skilled in the art will appreciate that the normally occurring amino acids at positions, for example, 13 (threonine), 121 (arginine), and/or 124 (tyrosine), may be shifted in the mutein. Thus, a change (mutation) of an amino acid to a cysteine residue at amino acid positions, for example, 38, 102 and/or 104 may be shifted on the mutein. However, the location of the shifted Ser (S), Arg (R), Tyr (Y) or Cys (C) can be determined by inspection and correlation of the flanking amino acids with those flanking Ser, Arg, Tyr or Cys in wtIL-4.

As used herein, the terms “mutant human IL-4 protein,” “modified human IL-4 receptor antagonist,” “mhIL-4,” “IL-4 mutein,” “IL-4 antagonist,” and equivalents thereof are used interchangeably and are within the scope of the invention. These polypeptides refer to polypeptides wherein specific amino acid substitutions to the mature human IL-4 protein have been made. As such, IL-4 muteins have 129 amino acid residues whose tertiary structure is a compact globular structure consisting of a four-helix bundle and have a predominantly hydrophobic core. As a result of E. coli-based expression all IL-4 muteins have an N-terminal methionine that remains as part of the purified proteins.

Such IL-4 muteins include, but are not limited to, the double mutein R121D/Y124D (“IL-4RA”):

(SEQ ID NO: 1)
M₋₁HKCDITLQEIIKTLNSLTEQKTLCTELTVTDIFAASKNTTEKETFCR
AATVLRQFYSHHEKDTRCLGATAQQFHRHKQLIRFLKRLDRNLWGLAGLN
SCPVKEANQSTLENFLERLKTIMDEKDSKCSS;

the triple mutein T13D/R121D/Y124D (“IL-4RA-T13D”):

(SEQ ID NO: 2)
M₋₁HKCDITLQEIIKDLNSLTEQKTLCTELTVTDIFAASKNTTEKETFCR
AATVLRQFYSHHEKDTRCLGATAQQFHRHKQLIRFLKRLDRNLWGLAGLN
SCPVKEANQSTLENFLERLKTIMDEKDSKCSS;

and the quadruple mutein T13D/N38C/R121D/Y124D (“IL-4RA-T13D-N38C”):

(SEQ ID NO: 3)
M₋₁HKCDITLQEIIKDLNSLTEQKTLCTELTVTDIFAASKCTTEKETFCR
AATVLRQFYSHHEKDTRCLGATAQQFHRHKQLIRFLKRLDRNLWGLAGLN
SCPVKEANQSTLENFLERLKTIMDEKDSKCSS.

The unpaired cysteine residue at position 38 in IL-4RA-T13D-N38C causes the renatured monomeric protein to be less stable at neutral pH prior to pegylation. Thus, IL-4RA-T13D-N38C contains seven (7) cysteine residues that form three (3) disulfide bond linkages. The N38C cysteine remains unpaired and is an exposed and reactive cysteine on the surface of the protein that can be used to attach a 20 kDa (linear or branched), a 30 kDa (linear), or a 40 kDa (linear or branched) polyethylene glycol (PEG) molecule. The fourth substitution, T13D, increases the potency of IL-4RA-T13D-N38C, so that after PEGylation IL-4RA-T13D-N38C is equipotent compared with IL-4RA. The theoretical isoelectric point (pI) for IL-4RA-T13D-N38C is 8.51, but this molecule can be expected to migrate to a position on an isoelectric focusing (IEF) gel to a pH greater than 9.3. (See, e.g., U.S. Pat. Nos. 6,028,176, 6,313,272, and 6,130,318, and U.S. Publication No. 20090010874, the entire content of each of which is incorporated herein by reference).

IL-4RA, IL-4RA-T13D, and IL-4RA-T13D-N38C bind with high affinity to the human IL-4 receptor alpha (IL-4Rα) chain, yet each has no ability to transmit a signal to intracellular pathways. Because both IL-4 and IL-13 receptor complexes require the participation of the IL-4Rα chain for effective signaling, IL-4RA, IL-4RA-T13D, and IL-4RA-T13D-N38C will block signaling associated with the binding of either cytokine to the human IL-4Rα chain. The IL-4 muteins have been evaluated for the treatment of asthma and atopic eczema, and they have been shown to effectively inhibit IL-4 and IL-13 mediated responses in in vitro systems of immune cell responses.

Methods for cell harvest, cell disruption, inclusion body purification, solubilization and chemical modification of SH-groups are well known procedures to those persons skilled in the art (Creighton T E (ed.) (1989): Protein structure—A Practical Approach. IRL Press, Oxford, N.Y., Tokyo). However, the present invention demonstrates that pH, temperature, and concentrations of sulfite and tetrathionate are critical factors affecting the quality of the sulfitolysis product. In addition, the number of diavolumes during the ultrafiltration/diafiltration (UF/DF) step of the solubilized sulfitolysized inclusion bodies (IBs) was also found to affect overall refold yield.

From the prior art it is well known that chemical agents of low molecular weight may suppress the formation of aggregates during refolding. Exemplary agents known for their ability to aid as aggregation suppressors during refolding, include, but are not limited to, L-arginine, urea, guanidinium hydrochloride, poly(ethylene)glycols, acetamide and short chain alcohols. However, most of these failed in case of Interleukin-4 derivatives and muteins with the exception of L-arginine and guanidinium hydrochloride.

Accordingly, the present invention provides a method for large-scale renaturation and recovery of proteins. The method includes adding to a refolding buffer containing H₂SO₄ or MgSO₄ in a Tris-base/Tris-HCl system, a solution of denatured, chemically modified or reduced protein in the presence of guanidine.

The denatured, chemically-modified, or reduced protein concentration in the diafiltration buffer may be about 8 gm/l or greater, about 10 gm/1 or greater, about 15 gm/l or greater, about 20 gm/l or greater, about 25 gm/l or greater, or about 30 gm/l or greater. Utilizing the methods of the invention, the concentration guanidine in the diafiltration can be reduced while recovering refolded protein in high yields and at high purity as described below. The guanidine concentration required may be less than about 8 M, less than about 7.5 M, less than about 7 M, less than about 6.5 M, less than about 6 M, less than about 5.5 M, less than about 5 M, less than about 4.5 M, less than about 4 M, less than about 4 M or less than about 3.5 M.

As provided in Example 3, the solubilization step releases the insoluble protein from its improperly folded and aggregated state in the IBs into a solution of soluble unfolded monomeric and polymeric proteins. In one embodiment, solubilization is initiated by addition of the IB Slurry to a solution containing 5 M to 9 M guanidine, 100-300 mM Tris, 1-7 mM EDTA, at pH 7 to pH 10 in a solubilization/sulfitolysis vessel, maintained at a temperature of 4 to 25° C., with mixing for a period of 1-24 hours. In another embodiment, solubilization is initiated by addition of the IB Slurry to a solution containing 7 M to 9 M guanidine, 100-300 mM Tris, 1-7 mM EDTA, at pH 7 to pH 9 in the solubilization/sulfitolysis vessel. In another embodiment, the solution contains 8 M guanidine, 200 mM Tris, 5 mM EDTA, at pH 9.0. In another embodiment the solution contains 8 M guanidine, 200 mM Tris, 5 mM EDTA at pH 7.5. Final solubilization conditions are typically 7 M guanidine, 175 mM Tris, 4.38 mM EDTA, at pH 7.5, maintained at 15-20° C. with mixing for a period of 1-24 hours. In yet another embodiment the solution contains 8 M guanidine, 200 mM Tris, 5 mM EDTA at pH 7.5. In yet another embodiment the final solubilization conditions are typically 7 M guanidine, 175 mM Tris, 4.38 mM EDTA, at pH 7.5, maintained at 18-22° C. with mixing for a period of 1 hour.

The solubilized IBs are then subjected to sulfitolysis, which generates a uniform solution of fully reduced monomeric protein by reducing existing disulfide bonds of proteins in the solubilized protein solution and modifying free sulfhydryls to form sulfocysteine residues. Solid sodium sulfite is added to the solubilized protein solution in a quantity sufficient to achieve a concentration of 5-30 gm sodium sulfite/L solubilized protein solution. This solution is allowed to mix slowly for 30-150 minutes. Solid potassium tetrathionate is then added to the solubilized protein/sodium sulfite solution in a quantity sufficient to achieve a concentration of 10-30 gm potassium tetrathionate/L solubilized protein. This solution is allowed to mix slowly for 30-150 minutes. In one embodiment, the solution is allowed to mix slowly for 30-150 minutes. In another embodiment, solid potassium tetrathionate is then added to the solubilized protein/sodium sulfite solution in a quantity sufficient to achieve a concentration of 10-30 gm potassium tetrathionate/L solubilized protein. This solution is allowed to mix slowly for 30-120 minutes. The sulfitolyzed protein solution is then pressure-fed through a dead-end filtration system and transferred to an ultrafiltration/diafiltration (UF/DF) hold vessel. In one embodiment, the filtration system includes more than one filter for serial filtration at decreasing pore size (e.g., 0.8 μm to 0.2 μm).

The sulfitolyzed protein is then subjected to ultrafiltration/diafiltration (UF/DF) to produce a post-diafiltration sulfitolyzed protein (PDSP). The purpose of this step is to concentrate the filtered sulfitolyzed protein and then diafilter the concentrated sulfitolyzed protein solution into the required buffer formulation for subsequent refold operations. A UF/DF system (skid) is set up to concentrate and diafilter the filtered sulfitolyzed protein solution. A clean UF/DF skid is typically equipped with an appropriate molecular weight cut-off filter and equilibrated with a solution. As discussed in Example 4, the UF/DF skid equipped with 10 kDa molecular weight cut-off filters is equilibrated with diafiltration buffer. The diafiltration buffer includes 3 M to 7 M guanidine, 25 mM to 75 mM Tris, and 1 mM to 7 mM EDTA, at pH 7.0 to 9.0 In one embodiment, the diafiltration buffer includes 4 M guanidine, 50 mM Tris, and 2 mM EDTA, at pH 9.0. In another embodiment, the difiltration buffer includes 4 M guanidine, 50 mM Tris, and 5 mM EDTA, at pH 9.0. In another embodiment, the difiltration buffer includes 4 M guanidine, 50 mM Tris, and 5 mM EDTA, at pH 7.5. In yet another embodiment, the diafiltration buffer may further comprise a chealating agent such as EDTA in a range of about 0.05 mM to about 100 mM or greater. The filtered sulfitolyzed protein is concentrated approximately 2 to 3-fold. The final target volume of the concentrated sulfitolyzed protein is referred to as the Final Retentate Volume.

The number of diavolumes was observed to have an affect on refold efficiency. It was hypothesized that a reduction of the number of diavolumes would significantly reduce the amount of guanidine consumed and would therefore be favorable if there was no impact to refold efficiency and other downstream processes. To test how many diavolumes are required to maintain refold efficiency, IBs were solubilized, sulfitolyzed, and filtered as above. The filtered material was concentrated to a protein concentration of approximately 20 mg/mL and diafiltered with a 4M guanidine diafiltration buffer. Samples were pulled after 0, 1, 3 and 5 diavolumes of buffer had been added. The protein concentrations in the PDSPs (post-diafiltration sulfitolyzed proteins) ranged from 16-17 gm/L. Refold was carried out in standard refold buffer with a final concentration of protein at 1.5 gm/L, with PDSP added to the refold mixture in 3 aliquots, 3 hours apart. Table 1 shows the total protein and active IL-4RA concentration after 24 hours of refold. The percent refold efficiency is the concentration of active IL-4RA measured by C4 RP-HPLC after refold divided by the total protein at start of refold (1.5 mg/mL).

TABLE 1
Effect of Number of Diavolumes on Refold Efficiency

| Number of | Total Protein | C4 RP-HPLC^{b} | | % Refold |
|---|---|---|---|---|
| Diavolumes | (mg/mL)^{a} | mg/mL | % Purity | Efficiency |
| 0 | 0.18 | 0 | 0 | 0 |
| 1 | 0.58 | 0.16 | 30 | 10.7 |
| 3 | 0.84 | 0.59 | 76.8 | 39.2 |
| 5 | 0.84 | 0.62 | 83.3 | 41.1 |

^{a}Total protein determined by Bradford assay as described in Example 1.
^{b}Concentration and purity of folded IL-4RA determined by C4 RP-HPLC as described in Example 1.

This experiment was also performed using PDSP that was made with more highly washed IBs, with the exception that the total concentration of protein in refold was 1 gm/L with PDSP added in 2 aliquots, 2.5 hours apart (Table 2).

TABLE 2
Effect of Number of Diavolumes on Refold Efficiency

| Number of | Total Protein | C4 RP-HPLC^{b} | | % Refold |
|---|---|---|---|---|
| Diavolumes | (mg/mL)^{a} | mg/mL | % Purity | Efficiency |
| 1 | 0.53 | 0.26 | 55.4 | 26.0 |
| 3 | 0.62 | 0.42 | 82.4 | 42.0 |
| 5 | 0.60 | 0.41 | 82.8 | 41.0 |

^{a}Total protein determined by Bradford assay as described in Example 1.
^{b}Concentration and purity of folded IL-4RA determined by C4 RP-HPLC as described in Example 1.

As such, in one embodiment, the protein is diafiltered with 1 to 10 diavolumes of a diafiltration buffer prior to addition to the refolding buffer. In another embodiment, the protein is diafiltered with 1 to 5 diavolumes. In yet another embodiment, the protein is diafiltered with 3 diavolumes of diafiltration buffer. The concentrated sulfitolyzed protein is diafiltrated against a volume of approximately three (3) to five (5) times the Final Retentate Volume of 4 M guanidine, 50 mM Tris, 5 mM EDTA, pH 9.0. The volume of the UF/DF hold vessel is maintained within a specified volume range of the Final Retentate Volume during the recirculation. After completion of the diafiltration of the concentrated sulfitolyzed protein the UF/DF system is flushed with 4M guanidine, 50 mM Tris, 5 mM EDTA, pH 9.0 into the UF/DF hold vessel.

The purpose of the refolding step is to refold (renature) sulfitolyzed denatured protein in the post-diafiltration sulfitolyzed protein (PDSP) solution (Final Diluted Retentate). As shown in Example 5, oxidative refolding is performed by dilution of the PDSP into the refolding buffer matrix containing 1M Tris-SO₄ with SO₄ concentrations ranging from 0 to 0.8M. As previously discussed, Tris-SO₄ is typically made, with H₂SO₄ and varying ratios of Tris Base/Tris HCl. The present invention utilized the prior art model to determine whether varying the concentration of SO₄ had an overall affect on refold efficiency. As such, H₂SO₄ and varying ratios of Tris Base/Tris HCl were combined to achieve a pH of 7.5. Concentrations of EDTA and cysteine remained constant at 5 mM and 1 mM, respectively. Table 3 lists the refold efficiency at varying SO₄ concentrations. Results show the optimal concentration of SO₄ ranges between 0.3 and 0.5M.

TABLE 3
Refold at Varying SO₄ Concentration

| | |
|---|---|
| C4 RP-HPLC^{a} | % Refold |

| [SO₄] M | mg/mL | % Purity | Efficiency |
|---|---|---|---|
| 0 | 0.29 | 69.3 | 29 |
| 0.1 | 0.33 | 72.3 | 33 |
| 0.2 | 0.35 | 74.9 | 35 |
| 0.4 | 0.38 | 77.0 | 38 |
| 0.6 | 0.36 | 80.1 | 36 |
| 0.8 | 0.28 | 73.6 | 28 |

^{a}Concentration and purity of folded IL-4RA determined by C4 RP-HPLC as described in Example 1

However, preparation of Tris-SO₄ by adding sulfuric acid to Tris base poses significant hazards at the manufacturing scale. As such, in one embodiment, Tris-hemisulfate is used as a substitute therefor. However, Tris-hemisulfate is manufactured in limited supply and is a substantially more expensive reagent than Tris Base and Tris HCl. Accordingly, in another embodiment, alternate non-limiting formulations, such as Tris plus SO₄²⁻ from (NH₄)₂SO₄, Na₂SO₄, and MgSO₄ may be used in the refold process. For example, Tris Base (0.125M) and Tris HCl (0.875M) may be added to 0.4 M sulfate salt (e.g., Na₂SO₄), 5 mM EDTA, 1 mM cysteine (final pH 7.5). Refold efficiency of the alternative sources of sulfate (SO₄²⁻) were then tested by adding protein PDSP to refold buffer in 2 aliquots of 0.75 g/L, 4 hours apart, and stirred for 24 hours at room temperature. The total protein content post refold was then measured. Results (Table 4) indicate that using Tris plus SO₄ from MgSO₄ is as effective as Tris-hemisulfate in refolding the protein.

TABLE 4
Alternative Formulations for Tris-SO₄

| | Total Protein | C4 RP-HPLC^{b} | | % Refold |
|---|---|---|---|---|
| Refold Condition | (mg/mL)^{a} | mg/mL | % Purity | Efficiency |
| 1 M Tris-hemisulfate | 0.65 | 0.50 | 79.2 | 33.3 |
| 1 M Tris-base/Tris HCl, | 0.43 | 0.36 | 80.5 | 24.0 |
| 0.4 M (NH₄)₂SO₄ | | | | |
| 1 M Tris-base/Tris HCl, | 0.53 | 0.42 | 79.0 | 28.0 |
| 0.4 M Na₂SO₄ | | | | |
| 1 M Tris-base/Tris HCl, | 0.65 | 0.52 | 76.1 | 34.7 |
| 0.4 M MgSO₄ | | | | |

^{a}Total protein determined by Bradford assay as described in Example 1.
^{b}Concentration and purity of folded IL-4RA determined by C4 RP-HPLC as described in Example 1.

In one embodiment, the refolding buffer comprises a source of sulfate ion in the range of about 0.01 to 0.8M, about 0.05 to 0.75M, about 0.1 to 0.7M, about 0.2 to 0.7M, about 0.3 to 0.7M, about 0.4 to 0.7M, about 0.1 to 0.6M, about 0.2 to 0.6M, about 0.3 to 0.6M, about 0.4 to 0.6M. The refolding buffer can further comprise a nonreducing sugar such as a polysaccharide, disaccharide, including sucrose, trehalose or other materials such as mannitol, in an amount of about 1% or greater, about 4% or greater, about 5% or greater, about 7% or greater, about 10% or greater, about 12% or greater, about 15% or greater, or about 17% or greater. The upper range of carbohydrate concentration (such as sucrose concentration) is about 30%. In some reactions, the upper concentration to still achieve desired results may be about 20% or about 25%. The refolding buffer can optionally further comprise a disulfide bond reducing agent such as beta-mercaptoethanol or the equivalent in a ranger of about 2 mM or greater, about 5 mM or greater, about 10 mM or greater, about 15 mM or greater, about 20 mM or greater, about 25 mM or greater, about 30 mM or greater, or about 40 mM or greater.

Thus, in another embodiment, the refold buffer of the invention includes 0.1 M to 0.6 M MgSO₄, 0.1 M to 0.6 M Tris Base, 0.4 M to 0.9 M Tris HCl, 2 mM to 7 mM EDTA, and 0.5 mM to 2 mM cysteine. In one embodiment, the refold buffer includes 0.2 M to 0.6 M MgSO₄, 0.1 M to 0.6 M Tris Base, 0.4 M to 0.9 M Tris HCl, 2 mM to 7 mM EDTA, and 0.5 mM to 2 mM cysteine. In another embodiment, the refold buffer includes 0.4 M MgSO₄, 0.125 M Tris Base, 0.875 M Tris HCl, 5 mM EDTA, and 1 mM cysteine. In another embodiment, the refold buffer further includes 0.25 mM beta-mercaptoethanol; and 2 mM cysteine HCl added just prior to start of PDSP refold.

Refold efficiencies achieved by the practice of the invention can be about 25% or greater, about 30% or greater, about 35% or greater, about 40% or greater, about 45% or greater, about 50% or greater, about 60% or greater, or about 70% or greater when carried out in refold reactor volumes of greater than about 200 liters. In some industrial scales, the refolding is carried out in volumes of greater than about 500 L, greater than about 1000 L, greater than about 2000 L, greater than about 4000 L, greater than about 5000 L, greater than about 7500 L, greater than about 8500 L, greater than about 10000 L, or greater than about 15000 L.

Refolding can be accomplished to produce protein concentrations of about 0.4 gm/L or greater, about 0.45 gm/L or greater, about 0.5 gm/L or greater, about 0.6 g/ml or greater, about 0.7 gm/L or greater, about 0.8 gm/L or greater, about 0.9 gm/L or greater, about 1 gm/L or greater, about 1.25 gm/L or greater, about 1.5 gm/L, about 2 gm/L, about 2.5 gm/L, or about 3 gm/L.

In another embodiment, the addition of excipients to the refold buffer, such as chemical chaperones, may inhibit formation of improperly folded intermediates of proteins and may promote the stabilization of folded intermediates of proteins and thus a reduction of aggregation (David, H. P.; Chemical chaperones: a pharmacological strategy for disorder of protein folding and trafficking. Pediatr. Res, 52 (2002): 832-836). Sugars and polymers can influence refolding by modifying diffusion rates and can stabilize proteins (Arakawa, et al.; Stabilization of protein-structure by sugars. Biochemistry, 21 (1982) 6536-6544; and Clark, et al., Inhibition of aggregation side reactions during in vitro protein folding, amyloid, prions, and other protein aggregates, Academic Press Inc., Orlando Fla., 1999, 217-236). Divalent cations have been shown to affect the refolding of some proteins (Singh, et al., Solubilization and refolding of bacterial inclusion body proteins, J. Biosci. Bioeng. 99 (2005) 303-310), with Mg²⁺ showing stabilizing effects with the protein during formulation studies. Amino acids, such as glycine have been shown to enhance stability of native structure (Serrano, et al. Effect of alanine versus glycine in alpha-helices on protein stability. Nature. 1992 Apr. 2; 356(6368):453-5).

For screening the effect of adding excipients to the refold buffer, 2 mL refold buffer was added to each well of a 24-well cell culture plate and IL-4RA PDSP added to a final concentration of 1.2 gm/L, using 2 additions 4 hours apart. The base refold buffer contained 1.0 M Tris-SO₄, pH 7.5, 5 mM EDTA, and 1 mM cysteine and served as a control. Study samples contained either 10 or 20% sucrose, 10 or 20% glycerol, 1 or 0.5% PEG 3350, 5 or 2.5% glycine, or 0.1 M MgCl₂, added to the base refold buffer. After refold for 24 hours, the samples were acidified, filtered and measured for total protein and correctly folded IL-4RA. Results indicate (not shown) that refold efficiency was unchanged (glycerol), reduced (PEG, glycine and MgCl₂), or slightly higher (sucrose) than the control. The MgCl₂ served to keep soluble a misfolded intermediate(s), as indicated by higher total protein and lower refolded IL-4RA than control samples. To further explore the effect of sucrose, 100 mL refold experiments were performed with sucrose concentrations ranging from 0 to 20%, and final concentration of PDSP added to refold at 1.5 and 2.0 gm/L. PDSP was added to refold buffer in 3 aliquots, 3 hours apart, and refold continued for a total of 24 hours before refold quench by acidification. Total protein and correctly folded IL-4RA were determined (Table 5) on the acidified, filtered refold.

TABLE 5
Sucrose addition to IL-4RA Refold Buffer

| | PDSP | Total | | | |
|---|---|---|---|---|---|
| | added | Protein | C4 RP-HPLC^{b} | | % Refold |
| % Sucrose | (gm/L) | (mg/mL)^{a} | mg/mL | % purity | Efficiency |
| 20 | 1.5 | 0.75 | 0.58 | 80.5 | 38.7 |
| 10 | 1.5 | 0.78 | 0.57 | 80.0 | 38.0 |
| 5 | 1.5 | 0.75 | 0.55 | 80.4 | 36.7 |
| 0 | 1.5 | 0.69 | 0.52 | 82.0 | 34.7 |
| 20 | 2.0 | 0.83 | 0.68 | 82.0 | 34.0 |
| 10 | 2.0 | 0.85 | 0.66 | 79.0 | 33.0 |
| 5 | 2.0 | 0.88 | 0.65 | 78.1 | 32.5 |
| 0 | 2.0 | 0.81 | 0.61 | 79.9 | 30.5 |

^{a}Total protein determined by Bradford assay as described in Example 1.
^{b}Concentration and purity of folded IL-4RA determined by C4 RP-HPLC as described in Example 1

Results show that adding 20% sucrose to the refold buffer led to an 11.5% increase of refolding efficiency; 10% sucrose yields a 9.6% increase; and 5% sucrose yields a 6% increase. Thus, in another embodiment, the refold buffer further contains about 5% to 20% sucrose. In another embodiment, the refold buffer contains 10% sucrose or 20% sucrose. In yet another embodiment, the refold buffer contains 20% sucrose.

Non-limiting examples of delivery of the PDSP to the refold buffer include single stream delivery, drop-wise delivery, pulsed delivery, and reverse dilution, with delivery time lasting approximately 30 minutes to 24 hours. As such, in one embodiment, the addition of PDSP to the refold buffer is by single stream with delivery time lasting approximately 30 min for a 3000 L refold. The time course of protein refold was then investigated. In one embodiment, PDSP is added to a final concentration of protein of about 0.5 to 3.0 gm/L and solution stirred for about 24 hours prior to harvest of the refold. In another embodiment, PDSP is added to a final concentration of about 0.5 to 2.0 g/L and solution stirred for about 24 hours prior to harvest of the refold

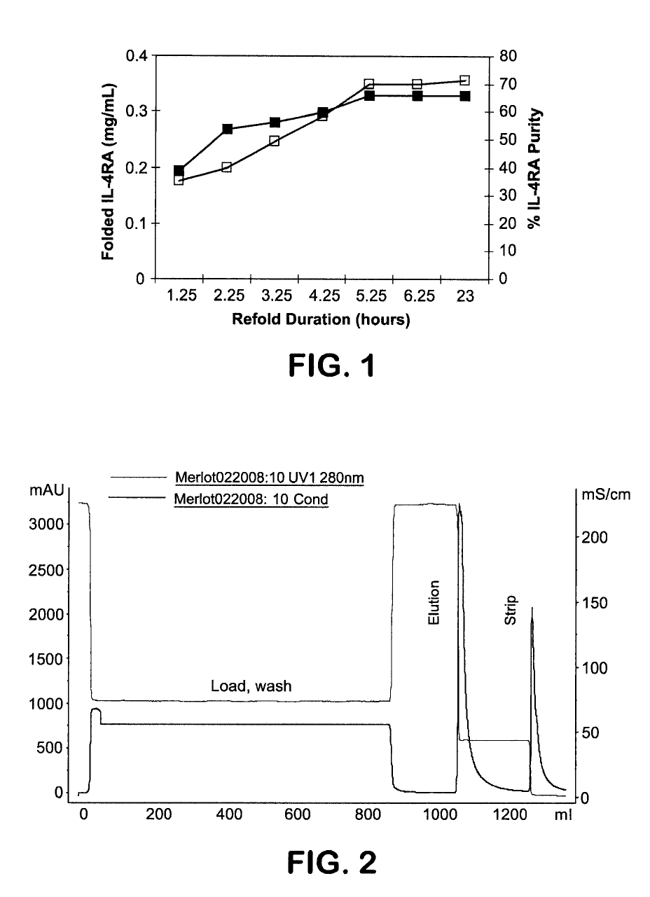

To determine the efficiency of refold when the protein is added in a single stream, IL-4RA PDSP was added to the standard refold buffer in a single pulse and samples were taken at every hour between 1.25 and 6.25 hours. Correctly folded IL-4RA and IL-4RA purity was measured by C4 RP-HPLC and results plotted in FIG. 1. As shown in FIG. 1, IL-4RA refold was initiated at T=0 and aliquots taken every hour for six hours. Refold continued for 23 hours and all samples measured for IL-4RA content (closed squares) and purity (open squares) by C4 RP-HPLC. Results show that there is an initial rapid increase in refolding rate from 0 to 2 hours, followed by a slower rate to a maximum at 6 hours. Protein purity, however, slowly increases over a 5-hour period.

In another embodiment, addition of the protein occurs via pulsed dilution. With pulsed dilution the denatured protein is added to the refold buffer in aliquots at specified times between additions. Allowing sufficient time between additions can avoid accumulation of high concentrations of folding intermediates (Tsumoto, et al. Solubilization of active green fluorescent protein from insoluble particles by guanidine and arginine. Biochem. Biophys. Res. Commun, 312 (2003a) 1383-1386). If the correctly folded structure does not aggregate with the unfolded or misfolded intermediates, a higher efficiency of refold may occur.

TABLE 6
Pulsed Delivery of Protein

| | | | Time | | | | |
|---|---|---|---|---|---|---|---|
| | PDSP | Number | between | Total | C4 RP-HPLC^{b} | | Refold |
| | added | of | addition | Protein | | Purity | Efficiency |
| Example No. | (gm/L) | additions | (hr) | (mg/mL)^{a} | (mg/mL) | (%) | (%) |
| 1 | 1.0 | 1 | 0 | 0.45 | 0.36 | 83.0 | 36 |
| 2 | 1.0 | 2 | 2.5 | 0.594 | 0.41 | 82.8 | 41 |
| 3 | 1.0 | 2 | 2.5 | 0.58 | 0.40 | 83.5 | 40 |
| 4 | 1.2 | 2 | 3 | 0.64 | 0.49 | 79.8 | 41 |
| 5 | 1.2 | 3 | 3 | 0.77 | 0.57 | 75.2 | 47 |
| 6 | 1.5 | 1 | 0 | 0.57 | 0.495 | 82.6 | 33 |
| 7 | 1.5 | 2 | 3 | 0.76 | 0.59 | 79.7 | 39.5 |
| 8 | 1.5 | 3 | 3 | 0.80 | 0.61 | 83.3 | 41 |
| 9 | 1.5 | 3 | 3 | 0.78 | 0.57 | 83.6 | 38 |
| 10 | 1.5 | 3 | 3 | 0.71 | 0.59 | 81.8 | 39 |
| 11 | 1.5 | 1 | 0 | 0.66 | 0.41 | 74.8 | 27 |
| 12 | 1.5 | 3 | 1 | 0.69 | 0.47 | 77.8 | 31 |
| 13 | 1.5 | 3 | 2 | 0.76 | 0.53 | 76.7 | 35 |
| 14 | 1.5 | 3 | 3 | 0.81 | 0.55 | 77.8 | 37 |
| 15 | 1.5 | 3 | 4 | 0.87 | 0.57 | 76.1 | 38 |
| 16 | 2.25 | 3 | 3 | 0.81 | 0.61 | 81.4 | 27 |
| 17 | 3.0 | 3 | 3 | 0.94 | 0.675 | 76.5 | 22.5 |
| 18 | 3.0 | 3 | 3^{c} | 0.95 | 0.65 | 74.7 | 21.7 |

^{a}Total protein determined by Bradford assay as described in Example 1.
^{b}Concentration and purity of folded IL-4RA determined by C4 RP-HPLC as described in Example 1.
^{c}The refold solution was filtered to remove insoluble aggregate between protein additions.

As shown in Table 6, pulsed dilution delivery allows for greater refold efficiency than protein delivery in a single pulse. Increasing the time between additions to 4 hours showed the greatest refold efficiency using 1.5 g/L denatured protein concentration. Filtering between additions provided no benefit, thereby demonstrating that correctly folded IL-4RA is not adversely affected by insoluble protein. Accordingly, exemplary pulsed dilutions include, but are not limited to 1 to 10 (e.g., 1, 2, 3, 4, 5, 6, 7, 8, 9, or 10) dilutions with 0 to 6 (e.g., 0, 1, 2, 3, 4, 5, or 6) hours between dilutions. Thus, in one embodiment, the protein is added in 1 to 3 dilutions with 0 to 4 hours between dilutions. In another embodiment, the protein is added in 3 dilutions with 4 hours between dilutions.

In another embodiment, addition of the protein occurs via reverse dilution. With reverse dilution, the refolding buffer is added to an unfolded protein resulting in a simultaneous decrease in both protein concentration and denaturant (i.e., guanidine). With mixing, refold buffer and unfolded protein are simultaneously delivered at a fixed ratio thereby maintaining constant denaturant and protein.

For example, the refold buffer is added to PDSP at a rate of 5 mL/min for a total of 20 minutes. For mixing, refold buffer was mixed with PDSP at a 10:1 ratio into a separate refolding container, for a duration of 10 minutes. Refolding by dilution with a single pulse of PDSP was used as a comparison. The initial protein concentration was 1.5 gm/L, with refold continuing for 24 hours prior to harvest under all conditions.

Known manufacturing procedures typically adjust the concentration of the PDSP to 10 gm/L prior to addition into the refold tank because higher concentrations cause mixing problems leading to increased aggregation during the initial phase of dilution. Surprisingly, the data presented herein demonstrates that the changes to the refold buffer discussed above allow higher concentrations of PDSP to be added to the refold tank. It was hypothesized that any increase in PDSP protein concentration would lead to a savings in guanidine consumption and reduce the carry over of guanidine into the refold tank.

As such, IL-4RA PDSP was concentrated to 15.8 and 35 g/L and stored at −20° C. prior to refold. PDSP was thawed at room temperature and added to standard refold buffer in a single addition to a final concentration of 1 gm/L. The 35 gm/L PDSP was added either as is or was first diluted 3-fold in 4M guanidine buffer prior to addition to refold. For comparison, Table 7 lists additional PDSPs used under identical refold conditions.

TABLE 7
Varying Concentration of PDSP

| | Concentration | | | C4 RP-HPLC^{a} | | |
|---|---|---|---|---|---|---|
| PDSP | of Stock PDSP | Refold | Volume of | | Purity | % Refold |
| No. | (g/L) | Reference | Refold (L) | (mg/mL) | (%) | Efficiency |
| 1a | 15.8 | 07 | 0.25 | 0.36 | 82.1 | 36 |
| 1b | 35.0 | 07 | 0.25 | 0.29 | 81.4 | 29 |
| 1c | 11.7^{b} | 07 | 0.25 | 0.36 | 83.4 | 36 |
| 1d | 11.7^{b} | 38 | 1.0 | 0.37 | 71.4 | 37 |
| 2a | 11.7^{b} | 99 | 3.0 | 0.35 | | 35 |
| 2b | 17.2^{b} | 151 | 3.5 | 0.34 | 83.0 | 34 |
| 3a | 20.1 | 186 | 4.0 | 0.36 | 85.4 | 36 |

^{a}Concentration and purity of folded IL-4RA determined by C4 RP-HPLC as described in Example 1
^{b}Diluted from 35 g/L stock prior to addition of refold

Results indicate that the PDSP can be stored at concentrations of at least 35 gm/L and can be delivered to the refold tank at concentrations ranging from 10 to 35 gm/L without observing a difference in refold yield or purity. Accordingly, in one embodiment, the protein is added to the refold buffer at a concentration of 10 to 35 gm/L. In another embodiment, the protein is added to the refold buffer at a concentration of 12 to 20 gm/L. In yet another embodiment, the protein is added to the refold buffer at a concentration of 20 gm/L.

Following the refold incubation period, the solution is prepared for capture and partial purification on a hydrophobic interaction chromatography (HIC) column. Hydrophobic interaction chromatography resins can be used in this invention to provide recoveries of refolded protein (optionally IL-4RA protein) from the refold buffer with efficiencies of about 60% or greater, about 65% or greater, about 70% or greater, about 75% or greater, about 80% or greater, about 85% or greater, about 90% or greater, or greater than about 95% when used in large scale batches of volumes greater than about 500 liters, as described earlier. The HIC resins used in carrying out the methods have a dynamic binding capacity of total protein (mg) to volume resin (mL) of greater than about 10 mg/mL, greater than about 12 mg/mL, greater than about 14 mg/mL, greater than about 16 mg/mL, greater than about 17 mg/mL, greater than about 18 mg/mL, or greater than about 20 mg/mL. The purity of recovered protein (optionally IL-4RA protein) is greater than about 75%, greater than about 80%, greater than about 85%, greater than about 90%, or greater than about 95%. The methods can be used on a commercial scale with recovered batch sizes of about 1.5 kg or greater, about 2 kg or greater, about 4 kg or greater, about 5 kg or greater, about 7 kg or greater, about 10 kg or greater, or about 20 kg or greater.

Accordingly, in one embodiment, sodium sulfate (Na₂SO₄) is added to a final concentration of 0.4 to 0.6 M to the refold hold vessel over a period of ≧1 hour to ensure the sodium sulfate went into solution, and after ensuring the sodium sulfate is fully dissolved, the pH of the refold solution is lowered to about 2.1 to 5.8 by addition of phosphoric acid (85%) to the refold hold vessel. In another embodiment, sodium sulfate is added to a final concentration of 0.6 M and the pH is lowered to 3.0±0.3. Once the pH of the refold solution in the refold hold vessel has been lowered to pH=3±0.3 and has stabilized, the pH-adjusted refold is immediately prepared for loading onto the HIC column. In yet another embodiment, the addition of the phosphoric acid lowering the pH to 3±0.3 is followed by addition of sodium sulfate to a final concentration of 0.4 M. The pH-adjusted refold must proceed directly to the depth filtration operation preceding the loading of the HIC column.

The refolded protein may be further purified from refold protein mixtures by either chromatographic or filtration methods. Application of numerous aspects of these methodologies are not practical in commercial-scale recovery and purification of refolded protein from the pH-adjusted refold matrix due to low capacity of protein binding, increased precipitation during ultrafiltration/diafiltration preparation for chromatography, low recoveries of refolded protein or the necessity for very cumbersome and logistically challenging manufacturing procedures. Only hydrophobic interaction chromatography (HIC) has been found to bind and concentrate the refolded protein and to remove residual product-related impurities (unfolded and misfolded protein) and host cell protein, endotoxin, and DNA impurities from the pH-adjusted refold matrix in a high-capacity process. A number of HIC resins were screened at both neutral and low pH conditions and many of these exhibit low binding capacity, which is not useful at large scale. Low capacity resins include Macro-Prep t-Butyl HIC (Biorad Laboratories), Octyl Sepharose 4 Fast Flow, Phenyl Sepharose 6 Fast Flow, Phenyl Sepharose HP, Butyl Sepharose 4 Fast Flow (GE Healthcare), and Super Butyl 5500 (Tosoh Biosciences). Exemplary resins for use in this high-capacity HIC method include, but are not limited to, Tosoh Biosciences Toyopearl Hexyl HIC, Toyopearl Butyl 650M, Toyopearl Butyl 600M, and GE Healthcare Capto Butyl resins. In certain embodiments, the resins may be packed in a 0.46×10 cm (Butyl 600M, Butyl 650M) or an 0.5×15 cm column (Capto Butyl) and equilibrated in buffer.

Hydrophobic charge induction chromatography (HCIC) resins were also screened for use at neutral pH and also found to exhibit low binding capacity. These resins include MEP hypercel and PPA Hypercel (Pall).

To determine dynamic binding capacities of the different resins, Tosoh Butyl 650M was compared to Butyl 600M (smaller pore size than the 650) and Capto Butyl resins from GE Healthcare. The resins were packed in either a 0.46×10 cm (Butyl 600M, Butyl 650M) or a 0.5×15 cm column (Capto Butyl) and equilibrated in buffer (Table 8). IL-4RA filtered refold was loaded onto the column at a flow rate of 150 cm/hr and flow-through fractions collected. Total protein of the flow-through fractions was monitored by Bradford and IL-4RA content was measured by C4 RP-HPLC. The dynamic binding capacity was calculated at 10% breakthrough of total protein.

TABLE 8
Dynamic Binding Capacity: Resin Screening

| | | | Dynamic Binding |
|---|---|---|---|
| | | | Capacity |
| | | Load Condition | Total Protein |
| Resin | Buffer A Condition | [Na₂SO₄] (M) | mg/mL Resin |
| Butyl 600 M | 2 M (NH₄)₂SO₄, 10 | 0.6 | 18.3 |
| | mM KPi, pH 3 | | |
| Butyl 650 M | 2 M (NH₄)₂SO₄, 10 | 0.6 | 14.8 |
| | mM KPi, pH 3 | | |
| Butyl 600 M | 1 M Na₂SO₄, 10 | 0.6 | 17.7 |
| | mM KPi, pH 3 | | |
| Capto Butyl | 2 M (NH₄)₂SO₄, 10 | 0.6 | 18.4 |
| | mM KPi, pH 3 | | |

Tosoh Toyopearl Butyl 600M had a 20% higher binding capacity than Butyl 650M, and a binding capacity equal to that of Capto Butyl. Changing the equilibration buffer to 1 M Na₂SO₄, 10 mM potassium phosphate (KPi), pH 3, the binding capacity changed slightly, but this may be due to differences in the refold material.

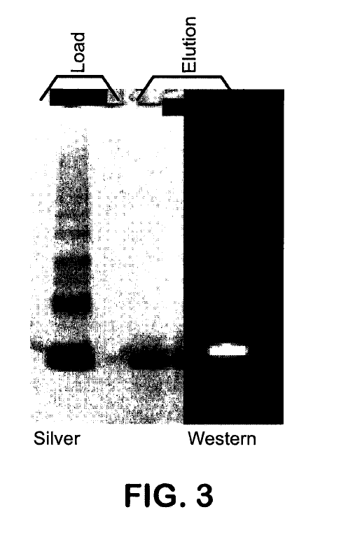

Tosoh Butyl 650M and Tosoh Butyl 600M were then scaled up for comparison of yield and purity at a target loading density of 11 g/L or 14 g/L, respectively, which represents a density of 75% of dynamic binding capacity. Butyl 650M was packed in equilibration buffer in a GE Heathcare XK 16 column to a bed height of 17 cm. Butyl 600M was packed in equilibration/wash buffer (2M (NH₄)₂SO₄, 10 mM potassium phosphate, pH 3.0) in a GE Heathcare XK 16 column to a bed height of 14 cm. For each chromatography run, the filtered refold (pH 3.0, 0.6M Na₂SO₄) was applied, the column washed with 5CV equilibration/wash buffer, eluted with 0.25 M (NH₄)₂SO₄, 10 mM potassium phosphate, pH 3.0 in 3 CV (main peak), 3 CV (peak tail), and stripped with 10 mM potassium phosphate, pH 3.0. The flow rate for each step was 150 cm/hr. Total protein was measured either by Bradford (refold, flow through and wash) or UV (elution and strip). Content of IL-4RA was measured by C4 RP-HPLC. The purity of the main elution was measured by C4 and C18 (Table 9). FIGS. 2 and 3 show the elution profile and SDS-PAGE results of the Butyl 650M run.

TABLE 9
Comparison of IL-4RA Recovery from Butyl 650M and Butyl 600M

| | Total | Total | IL-4RA Elution² | | |
|---|---|---|---|---|---|
| | Protein | IL-4RA | | % Purity | % Recovery |
| Resin | Load (mg)¹ | Load (mg)¹ | mg (C4) | (C18) | IL-4RA |
| Butyl 650M | 374 | 299 | 277 | 81.5 | 92.6 |
| Butyl 600M | 378 | 302 | 285 | 80.7 | 94.4 |

¹IL-4RA was refolded at 1 g/L, pH and salt adjusted, and filtered. The starting protein concentration was 0.43 mg/mL and IL-4RA concentration 0.344 mg/mL.
²IL-4RA eluted in the first 3CV's

The recovery and purity of IL-4RA was high for each of the Butyl 650M and Butyl 600M runs. Butyl 600M had a 20% higher capacity for the lot tested.

Capto Butyl and Butyl 600M were then chosen for scale up for comparison of yield and purity at a target loading density of 14 g/L or 12 g/L, respectively. Each resin was tested under two conditions to compare the performance of (NH₄)₂SO₄ to Na₂SO₄ in the equilibration, wash, and elution buffers. In each of the runs the filtered refold was applied at a flow rate of 150 cm/hr, washed for 5 column volumes, eluted in 3 column volumes (main), 3 additional column volumes (tail) and stripped with 10 mM potassium phosphate, pH 3.0. For chromatography in (NH₄)₂SO₄, the column conditions were as above. For Na₂SO₄ the equilibration and wash buffer consisted of 1 M Na₂SO₄, 10 mM potassium phosphate, pH 3.0, and the elution contained 0.2 M Na₂SO₄, 10 mM potassium phosphate, pH 3.0. Total protein was measured either by Bradford (refold, flow through and wash) or UV (elution and strip). Content of IL-4RA was measured by C4 RP-HPLC. The purity of the main elution was measured by C4 and C18. The total protein concentration of the load was 0.655 g/L and the IL-4RA concentration was 0.54 g/L. A target load density of 75% of dynamic binding capacity was chosen for each. Capto Butyl was packed to a higher bed height (22.5 cm) than the Butyl 600M (14.5 cm).

TABLE 10
IL-4RA Capture Chromatography on Butyl 600M and Capto Butyl: (NH₄)₂SO₄ Conditions

| | Elution 1 (1^{st} 3 CV)¹ | | | Elution 2 (2^{nd} 3CV) | | Strip | | |
|---|---|---|---|---|---|---|---|---|
| | % | % | % | % | % | % | % | % |
| | Recovery | Purity | Purity | Recovery | Purity | Recovery | Recovery | Purity |
| Resin | (C4) | (C4) | (C18) | (C4) | (C4) | (UV) | (C4) | (C4) |
| Butyl 600M | 86.1 | 95.4 | 74.4 | 0.56 | 27.7 | 13.3 | 0 | 0 |
| Capto Butyl | 77.5 | 99.4 | 83.3 | 3.3 | 95.6 | 1.9 | 1.2 | 93.3 |

¹Each resin was eluted with 0.25 M (NH4)2SO4, 10 mM potassium phosphate, pH 3.0

TABLE 11
IL-4RA Capture Chromatography on Butyl 600M and Capto Butyl: Na₂SO₄ Conditions

| | Elution 1 (1^{st} 3 CV)¹ | | | Elution 2 (2^{nd} 3 CV) | | Strip | | |
|---|---|---|---|---|---|---|---|---|
| | % | % | % | % | % | % | % | % |
| | Recovery | Purity | Purity | Recovery | Purity | Recovery | Recovery | Purity |
| Resin | (C4) | (C4) | (C18) | (C4) | (C4) | (UV) | (C4) | (C4) |
| Butyl 600M | 91.5 | 95.6 | 76.0 | 0.6 | 20.8 | 15.7 | 0 | 0 |
| Capto Butyl | 84.9 | 99.2 | 81.1 | 4.0 | 83.9 | 2.4 | 1.2 | 33.3 |

¹Each resin was eluted with 0.2 M Na2SO4, 10 mM potassium phosphate, pH 3.0

Butyl 600M had a higher recovery of IL-4RA in the 1st 3CV elution than the Capto Butyl in both the (NH₄)₂SO₄ (86.1% vs 77.5%) and Na₂SO₄ (91.5% vs 84.5%) conditions. However, the purity of the main eluted fractions were higher for the Capto Butyl resin. Capto Butyl also has the advantage that the resin can be packed to higher bed heights without an adverse effect on pressure (relative to Butyl 600M). In addition, Capto Butyl can be packed in 20% ETOH or water and does not shrink upon addition of high salt. Even though the Butyl 600M was packed in equilibration buffer, the column contracted when the refold was applied.

The Capto media is based on a high flow agarose matrix. Typical flow velocities at large scale (1 m diameter and 20 cm bed height) are 600 cm/hr with a back pressure below 1.5 bar. In contrast, the Tosoh Butyl 650M, a methacrylate based bead, operates at 150 cm/hr with a back pressure at 1.5 bar. As such, the operating time for Capto Butyl would be much less. IL-4RA recovery from Capto Butyl was tested using flow velocities ranging from 200 to 700 cm/hr, which represents contact times ranging from 1.24 min to 4.35 minutes. Results show (Table 12) that the recovery and purity of IL-4RA was independent of the contact time over the range studied.

TABLE 12
IL-4RA Recovery from Capto Butyl at Various Linear Flow Rates

| | Contact | | | |
|---|---|---|---|---|
| Linear Flow | Time | Load Density | % IL-4RA | % IL-4RA |
| rate (cm/hr) | (min) | (mg/mL Resin) | Recovery (C4) | Purity (C4) |
| 700 | 1.24 | 14.2 | 85.4 | 94.4 |
| 425 | 2.05 | 14.2 | 86.5 | 95.3 |
| 425 | 2.05 | 12.0 | 88.1 | 95.4 |
| 200 | 4.35 | 12.0 | 87.8 | 95.6 |

As discussed in Example 6, the pH-adjusted refold protein solution from the refold hold vessel is passed through one or more depth filters (e.g., 0.4 μm or 0.45 μm) followed by one or more polishing filters (e.g., 0.22 μm) before loading onto the column. In one embodiment, the filter is packed with diatomaceous earth and 1 M Na₂SO₄ is used as a loading buffer. The depth filter system is washed with purified water (WPU) until the effluent is clear. The refold hold vessel is then pressurized allowing the pH-adjusted refold protein solution to be filtered at an initial rate of ≦9.8 LPM (15.9 LPM maximum) and transferred to the filtered-refold hold vessel. Loading of the Toyopearl Butyl 650M HIC column (100×21 cm, 165 L) begins as soon as the filtered refold reaches a volume of approximately 800-1000 L in the filtered-refold hold vessel—target column loading density of about 7.5-9.3 gm/L total protein. In one embodiment, the target column loading density is about 10 gm/L total protein. A post-filtration flush of the depth filter system and polishing filter is performed with 2 M ammonium sulfate, 10 mM potassium phosphate, pH 3.0. In one embodiment the column is washed with equilibration buffer and the protein product is eluted with a conductivity step gradient of about 0.2 to 0.28 M ammonium sulfate, 8 mM to 12 mM potassium phosphate, pH 3.0. In another embodiment, the column is washed with equilibration buffer and the protein product is eluted with a conductivity step gradient of about 10-14% 2 M ammonium sulfate, 8 mM to 12 mM potassium phosphate, pH 3.0/85-90% 10 mM potassium phosphate, pH 3.0. In one embodiment, the protein product is eluted with a conductivity step gradient of 10-14% 2 M ammonium sulfate, 8 mM to 12 mM potassium phosphate, pH 3.0/85-88% 10 mM potassium phosphate, pH 3.0. In another embodiment, the protein product is eluted with a conductivity step gradient of 0.25 M ammonium sulfate, 10 mM potassium phosphate, pH 3.0. In another embodiment, the protein product is eluted with a conductivity step gradient of 12.5% 2 M ammonium sulfate, 10 mM potassium phosphate, pH 3.0/87.5% 10 mM potassium phosphate, pH 3.0. In another embodiment, the protein product is eluted with a lower conductivity step gradient of 10 mM potassium phosphate, pH 3.0.

The HIC eluate is collected when the ultraviolet (UV) monitor reaches 1.0%. HIC eluate collection continues until the UV monitor output returns to 1.0% or a 7-column volume elution volume is completed, whichever occurs first. Accordingly, the present invention also provides a method of isolating a refolded protein from a refolding matrix at a total protein concentration of about 0.4 to 2.0 gm/L. The method includes loading the pH-adjusted protein solution to a HIC column and eluting the protein with an elution buffer. In one embodiment, the protein product is eluted with a conductivity step gradient of 10-14% 2 M ammonium sulfate, 8 mM to 12 mM potassium phosphate, pH 3.0/85-88% 10 mM potassium phosphate, pH 3.0. In another embodiment, the protein product is eluted with a conductivity step gradient of 10-14% 2 M ammonium sulfate, 8 mM to 12 mM potassium phosphate, pH 3.0 and 90-86% 10 mM potassium phosphate, pH 3.0. In another embodiment, the protein product is eluted with a conductivity step gradient of 12.5% 2 M ammonium sulfate, 10 mM potassium phosphate, pH 3.0 and 87.5% 10 mM potassium phosphate, pH 3.0.

The purpose of this ultrafiltration/diafiltration (UF/DF) process step is to exchange the HIC eluate (12.5% 2 M ammonium sulfate, 10 mM potassium phosphate, pH 3.0/87.5% 10 mM potassium phosphate, pH 3.0) into an appropriate binding buffer (20 mM potassium phosphate, pH 6.1) for an optional next chromatography step (ceramic hydroxyapatite chromatography). An UF skid is set up to concentrate and diafilter the HIC eluate. A clean UF/DF skid is equilibrated in diafiltration buffer. In one embodiment, the diafiltration buffer includes 15 mM to 25 mM potassium phosphate at a pH=6.1±0.3. In another embodiment, the equilibration buffer includes 20 mM potassium phosphate, at pH 6.1. The eluate from the Butyl 650M chromatography step is concentrated to a single retentate at approximately 0 to 6 gm/L (e.g., 0.5 gm/L, 1.0 gm/L, 1.5 gm/L, 2.0 gm/L, 2.5 gm/L, 3.0 gm/L, 3.5 gm/L, 4.0 gm/L, 4.5 gm/L, 5.0 gm/L, 5.5 gm/L, and 6.0 gm/L) and diafiltration is performed with ≧5 times (e.g., 5, 6, 7, 8, 9, 10, or greater times) the retentate volume of 20 mM potassium phosphate, pH 6.1. In one embodiment, the eluate from the Butyl 650M chromatography step is concentrated to a single retentate at approximately 4.0 g/L and diafiltration is performed with ≧5 times the retentate volume of 20 mM potassium phosphate, pH 6.1. The diafiltered product is then flushed from the UF system with 15-25 L of the diafiltration buffer. In one embodiment, diafiltered product is then flushed from the UF system with 20 L of the diafiltration buffer. After a 10-15 minute recirculation, the entire flush volume is transferred into the UF/DF retentate hold vessel and mixed for not less than 5 minutes.

The following examples are intended to illustrate but not limit the invention.

### Example 1

### Analytical Assays

SDS-PAGE/Coommassie Blue—Denaturing, reducing polyacrylamide gel electrophoresis followed by Coomassie or Silver staining was used for determination of content and purity of the protein in PDSP, refold and purified samples. Samples were diluted in either 5M urea (PDSP) or H₂O (others), 4× NuPAGE® sample buffer and reducing agent, and heated to 70° C. for 10 minutes. Samples and the protein standard were loaded onto a 10 or 12-well NuPAGE® 4-12% Bis-Tris Gels (Invitrogen, 1 mm thickness) with MES running buffer under both reduced and non-reduced conditions. SDS-PAGE molecular weight standards (Mark 12, Invitrogen) were also loaded. The gels were run at 200 volts for about 40 minutes and then stained with either GelCode Blue COOMASSIE® Stain (Pierce Biotechnology) or SILVERXPRESS® Silver Staining Kit (Invitrogen) according to manufacturers instructions. Coomassie Stained Gels were imaged using Kodak Digital Image Stations 440_{CF} and band intensity measured. The content of the protein in PDSP was measured by comparing band intensity to the protein standard curve. Silver Stain Gels were imaged using Epson Perfection 1640SU document scanner.

C4 Reverse Phase—C4 RP-HPLC assay was used to distinguish the hydrophobic differences of the correctly folded molecule and improperly folded species in the refold and purified samples. Standard and samples were prepared by dilution into 0.1% TFA in water. A protein standard curve was generated by injecting 0.5, 1.25, 2.5, 5 and 7.5 μg of the protein onto a C4 RP-HPLC column (Vydac C4, 4.6×50 mm, 5 μm particle size) heated to 40° C. on an HP 1100 HPLC system (Agilent Technologies) at 1 mL/min. The HPLC solvents were 0.1% TFA in water (Solvent A) and 0.1% TFA in acetonitrile (Solvent B). Bound protein standard and protein samples were eluted with an acetonitrile gradient (Table 13) and the protein UV-monitored at 210 nm. The concentration of samples was determined from the peak area corresponding to the protein standard retention time. Percent purity was determined using the peak area corresponding to the protein standard retention time divided by the total peak areas.

TABLE 13
C4 RP-HPLC Gradient Program

| Time (min) | % B |
|---|---|
| 0.0 | 17.5 |
| 1.0 | 17.5 |
| 6.0 | 70.0 |
| 7.0 | 70.0 |
| 8.0 | 17.5 |
| 10.0 | 17.5 |

C18 In-Process—C18 RP-HPLC was used to distinguish the hydrophobic differences between the unmodified product and product related impurities including oxidized methionine, N-formyl methionine, isoleucine to β-Met Norleucine substitutions, and leucine to norvaline, among others. Standard and samples were prepared by dilution into 0.12% TFA in water to a concentration of 0.6 mg/mL and 50 μL injected onto an Agilent C18 RP-HPLC column, heated to 40° C. on an HP 1100 HPLC system (Agilent Technologies) at 0.5 mL/min. The HPLC solvents were 0.12% TFA in water (Solvent A) and 0.1% TFA in acetonitrile (Solvent B). Bound IL-4RA standard and IL-4RA column eluates were eluted with an acetonitrile gradient (Table 14) and the protein UV-monitored at 210 nm. The percent main peak purity was determined using the peak area corresponding to the protein standard retention time divided by the total peak areas.

TABLE 14
C18 In-Process RP-HPLC Gradient Program

| Time (min) | % B |
|---|---|
| 0.0 | 36 |
| 20.0 | 39 |
| 25.0 | 60 |
| 27.0 | 36 |
| 30.0 | 36 |

The C3 RP-HPLC assay can distinguish the hydrophobic differences of the correctly folded molecule and improperly folded species in the refold. Standard and samples were prepared by dilution into 0.1% TFA in water. An IL-4RA standard curve was generated by injecting 0.5, 1, 1.5, 2.5 and 5 μg AER 001 onto a C3 RP-HPLC column (Zorbax 300SB-C3, 4.6×50 mm I.D., 3.5 μm particle size, Agilent catalog No.: 5973-909) heated to 50° C. on an HP1100 HPLC system (Agilent Technologies) at 1 mL/min. The HPLC solvents were 0.1% TFA in water (Solvent A) and 0.1% TFA in acetonitrile (Solvent B). Bound IL-4RA standard, IL-4RA, and IL-4RA-T13D-N38C samples were eluted with an acetonitrile gradient (as shown below) and the protein was UV-monitored at 210 nm. The concentration of samples was determined from the peak area corresponding to the IL-4RA standard retention time. The % purity was determined using the peak area corresponding to the IL-4RA standard retention time divided by the total peak areas.

TABLE 15
C3 RP-HPLC Gradient Program

| Time (min) | % B |
|---|---|
| 0.0 | 30 |
| 2.0 | 30 |
| 12.0 | 65 |
| 13.0 | 2 |
| 14.0 | 2 |
| 15.0 | 30 |

Bradford—The Bradford assay, a Coomassie dye binding assay, was used to measure the concentration of total protein in PDSP and refold samples. Samples or BSA Standard (2 mg/mL Pierce) (20 μL each) were added to 380 μL Coomassie Plus™ Protein Assay Reagent (Pierce) to Row A of a 96-well plate and mixed well by pipetting up and down 8 times. A 2-fold dilution was performed by removing 200 μL from row A and adding to 200 μL Coomassie Protein Reagent in Rows B to G. Row H contained 200 μl, Coomassie Plus™ Protein Assay Reagent and used as a blank. Samples from PDSP were first diluted 10 fold with 4M guanidine, 50 mM Tris, pH 9.0, and 5 mM EDTA prior to addition to row A. Refold samples were added without adjustment. All samples and standards were run in duplicate. The plate was allowed to sit at room temperature for 10 minutes prior to reading on a Molecular Devices SPECTRA max PLUS Microplate spectrophotometer at 595 nm. The standard curve was fit to a 4-parameter fit and concentration of unknown samples determined using the SOFTmax PRO software.

### Example 2

### Preparation of Starting Materials

Cell lysis—At small scale, cells are suspended in Break Buffer containing 50 mM Tris, 2 mM EDTA, pH 7.5 and lysed using a Micofluidizer (Microfluidic Corp) by passing the cell suspension through three times at 10,000 to 15,000 psi. The machine and cooling coil chamber was packed with ice during the run.

Inclusion Body Washing—Inclusion bodies (IBs) were collected by centrifugation for 30 minutes at 6000 RPM. The inclusion body pellet was washed with 10× (wet weight) IB Wash Buffer containing 50 mM Tris, 2 mM EDTA, and 1% triton X-100, pH 7.5. For each cycle of washing, the pellet was resuspended in buffer and stirred on ice for 30 to 60 minutes to overnight at 4° C. prior to harvesting by centrifugation. A total of 3 washes were performed.

IB Slurry Preparation—A slurry was made by resuspending the final IBs in a volume (in milliliters) of IB Wash Buffer equivalent to the amount of grams wet-weight of the total IB pellet. The suspension was then homogenized by stirring and/or sonication.

### Example 3

### Inclusion Body Solubilization and Sulfitolysis

IL-4RA inclusion bodies (IB) were prepared in a slurry (35-50%) in 50 mM Tris, pH 7.5, 5 mM EDTA, 0.1% Triton X-100. Because there is variation in the slurry percentage in the aliquots, the slurry was first centrifuged for 20 minutes at 6000 RPM. The liquid was poured off, the weight of the IBs determined, and a 50% slurry made by addition of an equal amount of buffer (w/v) followed by complete resuspension. The 50% slurry was dissolved in a 7.5 fold volume of 8 M guanidine, 0.2 M Tris, pH 7.5 or 9, and 5 mM EDTA. Sodium sulfite was added to a concentration of 5 g/L and the solution stirred for 30 minutes at room temperature. Potassium tetrathionate was added to 10 g/L and the solution stirred for an additional 60 minutes to 3 hours. The mixture was filtered and stored at 4° C. for a maximum of 20 hour before ultrafiltration/diafiltration (UF/DF). The target protein concentration is 10 g/L, as determined by Bradford assay.

IL-4RA-T13D-N38C IBs were solubilized from a 35-50% slurry by addition of 7.5 times the volume of the IB slurry of solubilization buffer containing 8M guanidine, 20 mM Tris, and 2 mM EDTA, pH 7.5. Sodium sulfite (5 g/L) was added to the solubilization buffer prior to the addition of the 50% slurry of IBs. The Solubilization Buffer can be made without the addition of acid or base by using 80% Tris HCl and 20% Tris base. Potassium tetrathionate (10 g/L) was added to the mixture and the solution was stirred at room temperature for 60 minutes.

### Example 4

### Ultrafiltration/Diafiltration

The filtered solubilized protein was concentrated to approximately 20 g/L using three UF cassettes (0.1 sq meter, 10 kDa), with a feed pressure of approximately 15 psi and a retentate pressure of approximately 13 psi. After concentration, diafiltration buffer containing 4 M guanidine, 50 mM Tris, pH 7.5 or 9.0, and 5 mM EDTA was added at constant rate to maintain product concentration (IL-4RA); while diafiltration buffer containing 4 M guanidine, 20 mM Tris, and 1 mM EDTA, pH 7.5 (IL-4RA-T13D-N38C) was added to maintain product concentration. The post diafiltered sulfitolysized protein (PDSP) was collected after 5 diavolumes and then diluted to a final protein concentration (IL-4RA) of 10 g/L with 4M guanidine, 50 mM Tris, 2 mM EDTA, pH 7.5 or 9.0 generating the Final Diluted Retentate. Protein concentrations were determined by Bradford and PDSP stored in aliquots at −20° C.

### Example 5

### Refold

1 Liter IL-4RA Refold from PDSP Prepared at pH 7.5 and 9.0—Refold was performed by dilution of the PDSP in 3 equal aliquots, 3 hours apart into stirred refold buffer containing 1 M Tris-SO₄, 5 mM EDTA, and 1 mM cysteine, pH 7.5. The final concentration of added protein was 1.5 g/L. After continuous stirring for 24 hours at room temperature, the solution was adjusted to pH 3.0 by the addition of phosphoric acid. Sodium sulfate (Na₂SO₄) was added over a 30 minute period to achieve a final concentration of 0.6 M. Diatomaceous earth was added at 5 g/L to aid in filtration and the solution filtered through a 0.2 μm PES filter (Corning). The total protein was measured by Bradford, correctly folded protein measured by C4 RP-HPLC, and purity assessed by SDS-PAGE.

Stability of IL-4RA PDSP at pH 7.5 and 9.0, 100 mL Refolds—PDSP, prepared as described above at pH 7.5, was incubated at room temperature or −20° C. for 24 hours. A separate aliquot of the pH 7.5 PDSP was adjusted to pH 9.0 and incubated at room temperature or 40° C. for 24 hours. Refold was performed by dilution of PDSP into 100 mL stirred refold buffer in a single aliquot to achieve a total initial denatured protein concentration of 1.0 gm/L. After 24 hours of stirring, the refold was characterized by Bradford protein assay and C4 RP-HPLC.

1 L IL-4RA-T13D-N38C Refold from PDSP Prepared at pH 7.5—PDSP at 1 gm/L was refolded in Refold Buffer containing 0.875 M Tris-HCl, 0.125 M Tris-Base, 0.25 M MgSO₄-7H₂O pH 7.5; 0.25 mM beta-mercaptoethanol; and 2 mM cysteine HCl was added just prior to start of PDSP refold. A target time for the refold reaction was 4.5 hours at room temperature. The resulting product concentration was 135 μg/ml as determined by C3 HPLC and 315 μg/ml total protein as determined by Bradford protein assay using BSA as the standard.

Table 16 provides shows the results of a 1 liter refold. C3 HPLC is used to monitor the amount of correctly folded IL-4RA-T13D-N38C protein during the refolding process.

TABLE 16
IL-4RA-T13D-N38C Refold Results

| | | | Total | Capped | | PPI | Total | Total | | |
|---|---|---|---|---|---|---|---|---|---|---|
| | | Volume | protein | PPI | PPI | Conc. | PPI | Protein | % PPI | Process |
| Sample | Dilution | (ml) | (mA US) | (MA Us) | (mA US) | (μg/ml) | (mg) | (mg) | Purity | Yield % |
| PDSP | 1 | 44.8 | | | | | 0.0 | 1021.4 | | |
| 2.5 hr | 0.95 | 1040 | 17500.3 | 1784 | 4982 | 126.4 | 131.5 | 461.6 | 28.5% | 12.9% |
| refold | | | | | | | | | | |
| 3.5 hr | 0.95 | 1040 | 16138.9 | 1863.6 | 5307 | 134.6 | 140.0 | 425.7 | 32.9% | 13.7% |
| refold | | | | | | | | | | |
| 4.5 hr | 0.95 | 1040 | 15678.1 | 1978.3 | 5441 | 138.0 | 143.6 | 413.6 | 34.7% | 14.1% |
| refold | | | | | | | | | | |
| Pre- | 1 | 1050 | 14201.7 | 1971.6 | 5497.7 | 132.5 | 139.1 | 359.3 | 38.7% | 13.6% |
| filtered | | | | | | | | | | |
| Capto | 1 | 1050 | 7904.4 | 1316.7 | 3804.3 | 91.7 | 96.3 | 200.0 | 48.1% | 9.4% |
| Butyl | | | | | | | | | | |
| load | | | | | | | | | | |

| | | | | | |
|---|---|---|---|---|---|
| IL-4RA | 10 | 7350.5 | 6928.9 | 167.0 | RF = 41.5 |
| std | | | | | |

PPI = pre-PEGylated IL-4RA-T13D-N38C

### Example 6

### Capture Chromatography

IL-4RA Capture from IL Refolds—Tosoh Butyl 600M was packed in a XK16 column (GE Healthcare) to a bed height of 19 cm (38 mL bed volume) and equilibrated in buffer containing 2 M (NH₄)₂SO₄, 10 mM KPi, pH 3.0. Filtered refold was applied at a loading density of 14 g/L using a flow rate of 150 cm/hr, and the column washed with 5CV of equilibration buffer. The protein was eluted from the column with elution buffer containing 0.25 M (NH₄)₂SO₄ containing 10 mM KPi, pH 3.0, with a total of 3CV collected. The resin was subsequently stripped with H₂O, cleaned with 1 N NaOH followed by a H₂O rinse, and stored in 20% ETOH. The protein was measured by C4-HPLC and UV, purity was assessed by C18-IP and SDS-PAGE.

IL-4RA Capture from 100 mL Refolds—Tosoh Butyl 600M (3 mL packed volume) was added to each of 4, 10 mL empty columns (BioRad) and equilibrated with buffer as above. The entire 100 mL filtered refolds were applied in drip mode and the resin washed with 20 mL buffer. The protein was eluted from the column with 12 mL elution buffer. Purity of the protein was measured by SDS-PAGE.

IL-4RA-T13D-N38C Capture from IL Refolds—The 0.4M NaSO₄, pH 3.0 adjusted and filtered IL-4RA-T13D-N38C refold was loaded onto a Capto Butyl column that had been equilibrated in Buffer A (1 M Na₂SO₄, 20 mM Na₂PO₄, pH 4.0). The column was rinsed with Buffer A and the protein eluted with buffer B (20 mM Na₂PO₄, pH 4.0).

Although the invention has been described with reference to the above example, it will be understood that modifications and variations are encompassed within the spirit and scope of the invention. Accordingly, the invention is limited only by the following claims.

1. (canceled)

2. (canceled)

3. A method of isolating a refolded protein at a concentration of about 0.4 to 3 gm/L comprising:
a) loading a pH-adjusted protein solution to a hydrophobic interaction chromatography (HIC) column; and
b) eluting the protein with an elution buffer.

4. A method for large-scale renaturation of proteins comprising adding to a refolding buffer a protein solution of about 12 to 20 gm/L of denatured, chemically modified or reduced protein in the presence of guanidine, wherein the refolding buffer comprises MgSO₄ in a Tris-base/Tris-HCl system.

5. The method of claim 4, wherein the refolding buffer comprises 0.2 M to 0.6 M MgSO₄, 0.1 M to 0.6 M Tris Base, 0.7 M to 1.4 M Tris HCl, 2 mM to 7 mM EDTA, and 0.5 mM to 2 mM cysteine.

6. (canceled)

7. The method of claim 5, wherein the refolding buffer further comprises 0.1 mM to 0.4 mM beta-mercaptoethanol.

8. The method of claim 4, wherein the refolding buffer further comprises 5%-20% sucrose.

9. (canceled)

10. (canceled)

11. The method of claim 4, wherein the addition of the protein occurs via pulsed dilution.

12. The method of claim 11, wherein the protein is added in 1 to 3 dilutions with 0 to 4 hours between dilutions.

13. (canceled)

14. The method of claim 4, wherein the protein solution is diafiltered with 1 to 10 diavolumes of a diafiltration buffer prior to addition to the refolding buffer.

15. (canceled)

16. The method of claim 14, wherein the diafiltration buffer comprises 3 M to 7 M guanidine.

17. The method of claim 16, wherein the diafiltration buffer further comprises 25 mM to 75 mM Tris, and 1 mM to 7 mM EDTA, at pH 7.0 to 9.0.

18-21. (canceled)

22. The method of claim 4, further comprising recovering the protein via hydrophobic interaction chromatography (HIC).

23. The method of claim 22, wherein the pH is lowered to about 2.1 to 5.8 prior to HIC.

24. (canceled)

25. The method of claim 4, wherein the protein solution contains about 20 gm/L of protein.

26-29. (canceled)

30. A method for large-scale renaturation of proteins comprising:
a) diafiltering a denatured, chemically modified, or reduced protein with 1 to 10 diavolumes of a diafiltration buffer, wherein the diafiltration buffer comprises guanidine;
b) concentrating the diafiltered protein to about 12 to 20 gm/L; and
c) adding the concentrated protein via pulsed dilution to a refolding buffer, wherein the refolding buffer comprises MgSO₄ in a Tris-base/Tris-HCl system.

31. The method of claim 30, wherein the diafiltration buffer comprises 3 M to 7 M guanidine, 25 mM to 75 mM Tris, and 1 mM to 7 mM EDTA, at pH 7.0 to 9.0.

32-35. (canceled)

36. The method of claim 31, wherein the refolding buffer comprises 0.2 M to 0.6 M MgSO₄, 0.1 M to 0.6 M Tris Base, 0.7 M to 1.4 M Tris HCl, 2 mM to 7 mM EDTA, and 0.5 mM to 2 mM cysteine.

37. (canceled)

38. The method of claim 30, wherein the refolding buffer further comprises 0.25 mM beta-mercapto ethanol.

39. The method of claim 30, wherein the protein is diafiltered with 3 diavolumes of diafiltration buffer.

40. The method of claim 30, wherein the refolding buffer further comprises 5%-20% sucrose.

41. (canceled)

42. The method of claim 30, wherein the protein is added in 1 to 3 dilutions with 0 to 4 hours between dilutions.

43. (canceled)

44. The method of claim 30, further comprising recovering the protein via hydrophobic interaction chromatography (HIC).

45. The method of claim 30, wherein the pH of the mixture of the concentrated protein and the refolding buffer is lowered to about 2.1 to 5.8 prior to HIC.

46. (canceled)

47. The method of claim 30, wherein the solution contains about 20 gm/L of protein.

48-52. (canceled)

53. The method of claim 3, wherein the elution buffer comprises 10-14% 2 M ammonium sulfate, 8 mM to 12 mM potassium phosphate, pH 3.0/85-88% 10 mM potassium phosphate, at pH 3.0.

54. (canceled)

55. The method of claim 3, wherein the protein solution is adjusted to a pH of about 2.1 to 5.8 prior to HIC.

56-59. (canceled)


SEQUENCE LISTING (3 sequences)

SEQ ID NO: 1 - protein, 130 aa, Artificial Sequence
  Synthetic construct
MHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMDEKDSKCSS

SEQ ID NO: 2 - protein, 130 aa, Artificial Sequence
  Synthetic construct
MHKCDITLQEIIKDLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMDEKDSKCSS

SEQ ID NO: 3 - protein, 130 aa, Artificial Sequence
  Synthetic construct
MHKCDITLQEIIKDLNSLTEQKTLCTELTVTDIFAASKCTTEKETFCRAATVLRQFYSHHEKDTRCLGATAQQFHRHKQLIRFLKRLDRNLWGLAGLNSCPVKEANQSTLENFLERLKTIMDEKDSKCSS